(19) United States
(12) Patent Application Publication
(10) Pub. No.: US 2023/0311101 A1
(43) Pub. Date: Oct. 5, 2023

(54) PROCESS FOR THE DIRECT CONVERSION OF CELLULOSE TO GLYCOLS USING NON-NOBLE METAL LOADED ZEOLITE CATALYSTS

(71) Applicant: COUNCIL OF SCIENTIFIC & INDUSTRIAL RESEARCH, New Delhi (IN)

(72) Inventors: Banu MARIMUTHU, Pune (IN); Sreejith SREEKANTAN, Pune (IN); Arun Arunima Balachandran KIRALI, Pune (IN)

(21) Appl. No.: 18/022,855

(22) PCT Filed: Aug. 24, 2021
(86) PCT No.: PCT/IN2021/050808

§ 371 Date: Feb. 23, 2023

(30) Foreign Application Priority Data
Aug. 24, 2020 (IN) 202011036465

(51) Int. Cl. B01J 23/888; B01J 29/48; B01J 37/04; B01J 37/02; B01J 37/00; B01J 37/08; B01J 37/18; C07C 29/132; C07H 3/02; C07C 29/60; C07H 1/00

(52) U.S. Cl. CPC B01J 23/888; B01J 29/48; B01J 37/04; B01J 37/0236; B01J 37/0036; B01J 37/088; B01J 37/18; C07C 29/132; C07H 3/02; C07C 29/60; C07H 1/00

(57) ABSTRACT

The present invention relates to a process for the direct conversion of cellulose into glycols by using a non noble metal supported zeolite catalyst selected from Al—Ni—W/HY, Al—Ni—W/NaY and Al—Ni—W/Na-ZSM-5, wherein the ratio of the metal in the catalyst is in the range of 15%-12%-30% to 0%-3%-5%.

### FIELD OF THE INVENTION

The present invention relates to a process for the direct conversion of cellulose into glycols by using metals supported on zeolite catalysts. Particularly, the present invention relates to a process for the direct conversion of cellulose into glycols by using non-noble metal supported zeolite catalysts.

### BACKGROUND OF THE INVENTION

The most abundant source of biomass is cellulose, which is now a days regarded as a promising alternative for fossile fuels as it cannot be digested by human beings. Catalytic conversion of cellulose to glycol has much attention in recent years. Glycols are very valuable products considering their applicability in various industrial areas. They are useful compounds in the manufacturing of polyesters, particularly PET (polyethylene terephthalate), which is used widely for clothes and packaging.

The article entitled “Catalytic conversion of cellulose for efficient ethylene glycol production and insights into the reaction pathways” by Kai Zhang et. al and published in the journal “RSC Adv., 2016, 6, 77499-77506” reports the use of tungsten-containing heteropoly acids with the combination of supported noble metals forone-pot hydrothermal conversion of cellulose into polyols in the presence of pressurized hydrogen. It describes that a microcrystalline cellulose was completely converted over a mixed catalyst consisting of a low concentration of phosphotungstic acid (PTA) (0.03 wt %) and Ru/activated carbon (Ru/AC) via an one-pot hydrothermal reaction, with an ethylene glycol (EG) yield up to 53.1% under optimal conditions.

The article entitled“Cellulose conversion to polyols on supported Ru catalysts in aqueous basic solution” by Sun Jiying and Liu Haichao and published in the journal “Science China Chemistry volume 53, pages 1476-1480 (2010)” reports selective conversion of cellulose into ethylene glycol, propylene glycol with supported Ruthenium catalyst. The article reports that ethylene glycol, 1,2-propanediol and 1, 2, 5-pentanetriol were obtained with selectivities of 15%, 14% and 22%, respectively at 38% cellulose conversion at pH 8 in phosphate buffer solution.

So far the reported catalysts for the synthesis of glycols are precious-metal catalysts which are too expensive and less stable. Therefore, it is highly desirable to develop a less expensive but efficient catalyst to replace precious-metal catalysts in this cellulose degradation process.

The catalysts known in the art involves tedious synthetic process and are unstable in nature. Hence, they are not useful for commercialization. Thus, there is a need in the art to provide a catalyst system which will give high conversion of cellulose into valuable products.

### Objectives of the Invention

Main objective of the present invention is to provide a process for the conversion of cellulose into polyols by using non-noble metal supported zeolite catalysts.

Another objective of the present invention is to provide a cost-effective, environmental friendly, stable and reusable catalyst for the efficient conversion of cellulose to glycols.

Another objective of the present invention is to obtain a 100% conversion of cellulose into valuable products such as ethylene glycol and 1, 2 propane diol with highest selectivity.

### SUMMARY OF THE INVENTION

Accordingly, the present invention provides a process for conversion of cellulose into polyols comprising the steps of:
i. reacting cellulose with a catalyst in the ratio ranging between 1:0.3 to 1:1 in a batch reactor with a solvent, at a temperature in the range of 200° C. to 230° C., pH in the range of 4.5 to 2 with stirring in the range of 700 to 1000 rpm, and pressure in the range of 40 to 70 bars of Hydrogen for a period in the range of 1.5-12 hrs to obtain polyol with 100% conversion of cellulose to polyols;
wherein
the polyol is ethylene glycol, 1,2-propane diol, glucose, sorbitol, xylitol and glycerol;
the catalyst is selected from the group consisting of Al—Ni—W/HY, Al—Ni—W/NaY Al—Ni—W/HZSM-5 and Al—Ni—W/Na7SM-5.

In an embodiment of the present invention, the ratio of the metal in said catalyst is Ni in the range of 3-12%, Al in the range of 0-15% and W in the range of 5-30%.

In an embodiment, the present invention provides a catalyst for 100% conversion of cellulose in polyol comprising:
i. Al—Ni—W, wherein the Al is in the range of 0%-15%; Ni is in the range of 3%-12%; and W is in the range of 5%-30%;
ii. a support adding up to 100% wherein the support is selected from HZSM-5, Na-ZSM-5, Y or beta zeolite; HY zeolite, NaY Zeolite, gamma Alumina or boehmite.

In another embodiment of the present invention, the catalyst 5% Al-8% Ni-25% W/Na-ZSM-5 provides ethylene glycol (EG) selectivity of 89-92.1%.

In still another embodiment of the present invention, the catalyst 5% Al-8% Ni-25% W/Na-ZSM-5 is recyclable.

In an embodiment, the present invention provides a process for preparation of catalyst, wherein said catalyst is prepared by wet impregnation method comprising the steps of:
a) simulataneously adding solutions of metal precursors in a solvent on a support under stirring at a temperature in the range of 60-85° C. for a period of time in the range of 6-8 hrs to obtain a first mixture;
b) drying the first mixture as obtained in step (a) in an oven at a temperature in the range of 80-120° C. for a period of time in the range of 10-12 hrs to obtain a second mixture;
c) grinding and calcining the second mixture as obtained at step (b) at a temperature in the range of 500-600° C. for a period in the range of 4 to 5 hrs followed by reducing under hydrogen at a temperature in the range of 350-450° C. for a period in the range of 4 to 5 hrs to obtain the catalyst.

In yet another embodiment of the present invention, the support in step (a) is selected from the group consisting of ZSM-5, Na-ZSM-5, Y or beta zeolite; HY zeolite, NaY Zeolite, gamma Alumina or boehmite, preferably ZSM-5 or Y.

In yet another embodiment of the present invention, said metal precursors in step (a) are Aluminium nitrate nonahydrate, Ammonium metatungstate and Nickel nitrate hexahydrate.

In yet another embodiment of the present invention, cellulose is selected from pure cellulose or bio-cellulose.

In yet another embodiment of the present invention, the solvent is water.

### BRIEF DESCRIPTION OF THE DRAWINGS

FIG. 1 depicts HPLC chromatogram for various value added products for the active catalyst (5% Al-8% Ni-25% W/ZSM-5) at 40 bar pressure H₂ (at 25° C.), 12 hours, 220° C. temperature reaction conditions

FIG. 2 depicts the influence of Ni for cellulose conversion and glycol yields over W/NaY catalyst at 30 bar pressure H₂ (at 25° C.), 6 hours and 220° C. temperature reaction conditions.

FIG. 3 depicts the influence of Ni for cellulose conversion and glycol yields over W/HY catalyst at 30 bar pressure H₂ (at 25° C.), 6 hours and 220° C. temperature reaction conditions.

FIG. 4 depicts the influence of different metal loaded on NaY support for cellulose conversion and glycol yields over 30 bar pressure H₂ (at 25° C.), 3 hours and 220° C. temperature reaction conditions.

FIG. 5 depicts the influence of Ni for cellulose conversion and glycol yields over W/HY catalyst at 30 bar pressure H₂ (at 25° C.), 3 hours and 220° C. temperature reaction conditions.

FIG. 6 depicts the cellulose conversion and the glycol yields over NaY support at 40 bar pressure H₂ (at 25° C.), 6 hours and 220° C. temperature reaction conditions.

FIG. 7 depicts the cellulose conversion and the glycol yields over HY support at 40 bar pressure H₂ (at 25° C.), 6 hours and 220° C. temperature reaction conditions.

FIG. 8 depicts the cellulose conversion and the glycol yields over NaY support at 40 bar pressure H₂ (at 25° C.), 3 hours and 220° C. temperature reaction conditions.

FIG. 9 depicts the cellulose conversion and the glycol yields over NaY support at 30 bar pressure H₂ (at 25° C.), 3 hours and 220° C. temperature reaction conditions.

FIG. 10 represents XRD of 5% Al-8% Ni-25% W/Na7SM-5 before reaction and after 4 cycles.

FIG. 11 represents HR-TEM images of spent catalyst after 4 cycles.

FIG. 12 represents (a) FE-SEM of synthesized Na7SM-5 zeolite; and (b) FE-SEM of spent catalyst after 4 cycles.

### DETAILS OF THE BIOLOGICAL RESOURCES USED

Pure Cellulose used in example 2 was obtained from Sigma Aldrich.

Sugarcane bagasse used in example 3 was obtained from Local Vendor, NCL Shopping Complex, CSIR-National Chemical Laboratory, Dr. Homi Bhabha Road, Pune 411008.

### DETAILED DESCRIPTION OF THE INVENTION

The present invention provides a process for the direct conversion cellulose into polyols by using non-noble metal supported zeolite catalyst, wherein the process comprises of reacting cellulose with a catalyst in the ratio of 1:0.3 to 1:1, wherein catalyst is selected from the group consisting of Al—Ni—W/HY, Al—Ni—W/NaY and Al—Ni—W/Na-ZSM-5 in the ratio of Ni varying from 3-12%, Al varying from 0-15% and W varying from 5-30% at a temperature in the range of 200° C. to 230° C., pH in the range of 4.5 to 2, stirring in the range of 700 to 1000 rpm, and pressure in the range of 30 to 70 bars of hydrogen, to obtain 100% conversion of cellulose resulting in the polyols.

The process is carried out in Parr reactor with water as a solvent.

The polyol is selected from the group consisting of ethylene glycol, 1,2-propane diol, glucose, sorbitol erythritol, propanol and ethanol, with selectivity of total glycols ranging from 56.9% to 92.8% all adding up to 100%. The solid products are separated, dried and weighed to find out the conversion. The difference in the weight of substrate before and after reaction is used to calculate the Conversion.

If Cᵢ is the initial amount of cellulose and C_{f} is the final amount of cellulose, considering that all catalyst is recovered;

C_{f}=Amount of solid products after reaction−Amount of catalyst initially used

Conversion (%)=((Cᵢ−C_{f})/Cᵢ)×100

Microcrystalline cellulose powder (20 μm) used in the conversion is purchased from Sigma Aldrich. However, the efficiency of the catalyst has also been studied on higher concentration of cellulose (1 wt % to 5 wt %) and real source biomass. The real source biomass is pretreated sugarcane bagasse, wherein the pre treatment is well established processes to separate the cellulosic components from the ligno cellulosic and hemi cellulosic components. These studies show that the conversion and yield are good even at lower cellulose to catalyst ratio.

Suitable temperature to conduct the conversion reaction is in the range of 200° C. to 230° C.

The temperature is 220° C. in the view of mild reaction conditions. Suitable pressure to conduct the conversion reaction is in the range of 30 to 70 bars of Hydrogen (at 25° C.). The pressure is 40 bar at the 25° C., reaching up to 70 bar at reaction temperature of 220° C. Suitable pH to maintain at the reaction is in the range of 4.5 to 2.

The present invention provides a cost-effective, environmental friendly, stable and reusable catalyst for the efficient conversion of cellulose to glycols, wherein the above said catalyst is selected from Al—Ni—W/HY, Al—Ni—W/NaY and Al—Ni—W/Na-ZSM-5 with the ratios of Ni varying from 3-12%, Al varying from 0-15% and W varying from 5-30%.

The present invention provides a wet impregnation process for the preparation of the catalyst, wherein the process for the preparation of the catalyst comprises of adding precursor metal solutions in a suitable solvent simultaneously on a support under stirring at a temperature in the range of 60-85° C. for a period of time in the range of 6-8 hrs; drying the obtained mixture in an oven at a temperature in the range of 80-120° C. for a period of time in the range of 8-12 hrs; followed by grinding and calcining at a temperature in the range of 500-600° C. for 4-6 hrs and reducing under hydrogen at a temperature in the range of 350-450° C. for a 4-6 hrs to obtain the catalyst.

Zeolites/supports used in the preparation of the catalyst for the direct conversion are selected from ZSM-5, Na-ZSM-5, Y or beta zeolite or gamma Alumina or boehmite, preferably ZSM-5 or Y.

The choice of the support is based on acidity, specific structure, high thermal and hydrothermal stability.

The nickel is used for its hydrogenation ability. Compared to other hydrogenation metals, nickel is non noble metal and comparatively less expensive. The tungsten metal is well studied for the C—C bond cleavage property and facilitates retro aldol condensation fairly well. Since the hydrolysis of cellulose is the very basic step of cellulose conversion, aluminium was tried as if it could efficiently increase the hydrolysis.

The Al concentration is varied from 0-15%. Ni concentration is varied from 3-12% and W concentration is varied from 5-30%. Based on the variation in metal loading, the amount of metal precursor used has also been calculated accordingly.

Metal precursors Aluminium nitrate nonahydrate (˜98%, Thomas Baker), Ammonium metatungstate (˜99.99%, Aldrich Chemistry), Nickel nitrate hexahydrate (˜99.999%, Aldrich chemistry) are used for the catalyst preparation.

Non-noble metals loaded on the support in a catalyst are selected from Al (helps in hydrolysis), Ni (for hydrogenation), W (for selective cleavage and retro aldol reaction).

Suitable solvent to dissolve the precursor are selected from a polar protic solvent. The polar protic solvent may include water, methanol, or ethanol. Preferably water is used as a solvent.

The present invention provides a catalyst, reusable for consecutive runs without any change in structural properties of support of leaching of metals.

Several experiments have been conducted at different reaction conditions (temperature, pressure, catalyst amount and reaction time) by using different catalyst system for cellulose conversion. The results are summarized below in Table 1. The pressures given in the table are the pressure at 25° C.

TABLE 1

| | | | Selectivity (% ) | | |
|---|---|---|---|---|---|
| | | | | | Total |
| Sr. | | Conversion | | | glycol |
| No | Catalysts | (%) | EG | 1, 2 PD | (%) |
| 1 | 8% Ni-15% W/NaY | 81 | 43.93 | 18.25 | 62.15 |
| | 220° C., 35 bar, 6 hrs, | | | | |
| | 1000 rpm; | | | | |
| 2. | 8% Ni-15% W/NaY | 96.71 | 46.99 | 16.82 | 63.81 |
| | 220° C., 40 bar, 6 hrs, | | | | |
| | 1000 rpm; | | | | |
| 3. | 8% Ni-15% W/NaY | 95.04 | 36.61 | 15.12 | 51.73 |
| | 220° C., 30 bar, 6 hrs, 1000 rpm | | | | |
| 4. | 5% Al-8% Ni-15% W/NaY; | 98 | 44.64 | 16.5 | 61.14 |
| | 220° C., 40 bar, 6 hr, 1000 rpm | | | | |
| 5. | 15% Al-6% Ni-10% W/NaY; | 100 | 31.56 | 19.72 | 51.28 |
| | 220° C., 40 bar, 6 hr, | | | | |
| | 1000 rpm | | | | |
| 6. | 5% Al-6% Ni-15% W/NaY | 96.023 | 35.84 | 17.09 | 52.93 |
| | 220° C., 30 bar, 6 hr, 1000 rpm | | | | |
| 7. | 5% Al-6% Ni-15% W/NaY | 78.6 | 32.06 | 11.06 | 43.12 |
| | 220° C., 30 bar, 3 hr, 1000 rpm | | | | |
| 8 | 5% Al-6% Ni-10% W/NaY | 91.8 | 22.78 | 42.38 | 65.16 |
| | 220° C., 30 bar, 3 hr, 1000 rpm | | | | |
| 9. | 5% Al-6% Ni-10% W/NaY | 68.95 | 22.11 | 19.25 | 41.36 |
| | 220° C., 40 bar, 3 hr, 1000 rpm | | | | |
| 10. | 10% Al-6% Ni-10% W/NaY; | 95.8 | 42.2 | 24.96 | 67.16 |
| | 220° C., 40 bar, 6 hr, 1000 rpm | | | | |
| 11. | 5% Al-8% Ni-20% W/NaY; | 100 | 50.08 | 14.08 | 64.26 |
| | 220° C., 40 bar, 6 hr, 1000 rpm | | | | |
| 12. | 8% Ni-20% W/NaY; | 100 | 27.76 | 24.81 | 52.57 |
| | 220° C., 40 bar, 6 hr, 1000 rpm | | | | |
| 13. | 5% Al-6% Ni-15% W/NaY | 98 | 50.19 | 19.40 | 69.59 |
| | (1:1) 220° C., 40 bar, 6 hrs, | | | | |
| | 1000 rpm | | | | |
| 14. | 5% Al-8% Ni-20% | 100 | 52.04 | 14.91 | 66.95 |
| | W/NaY(1:1); 220° C., | | | | |
| | 40 bar 6 hrs, 1000 rpm | | | | |
| 15. | 5% Al-8% Ni-25% W/NaY; | 100 | 48.91 | 12.06 | 60.97 |
| | 220° C., 40 bar 6 hrs, 1000 rpm | | | | |
| 16. | 5% Al-8% Ni-20% W/HY | 100 | 62.79 | 11.78 | 74.57 |
| | 220° C., 40 bar 6 hrs, 1000 rpm | | | | |
| 17. | 5% Al-6% Ni-10% | 100 | 63.88 | 3.75 | 67.63 |
| | W/NaZSM-5; 220° C., | | | | |
| | 40 bar 6 hrs, 1000 rpm | | | | |
| 18. | 8% Ni-20% W/NaZSM-5; | 100 | 53.44 | 3.50 | 56.94 |
| | 220° C., 40 bar 6 hrs, 1000 rpm | | | | |
| 19. | 5% Al-8% Ni-25% | 100 | 73.31 | 8.94 | 82.25 |
| | W/NaZSM-5; 220° C., | | | | |
| | 40 bar 6 hrs, 1000 rpm | | | | |
| 20. | 5% Al-8% Ni-25% | 100 | 76.71 | 7.54 | 84.25 |
| | W/NaZSM-5 (1:1); 220 b, | | | | |
| | 40 bar 6 hrs, 1000 rpm | | | | |
| 21. | 5% Al-8% Ni-25% | 100 | 61.85 | 8.34 | 70.19 |
| | W/NaZSM-5; 220° C., | | | | |
| | 40 bar , 1.5 hrs, 1000 rpm | | | | |
| 22. | 5% Al-8% Ni-25% | 100 | 62.56 | 19.42 | 81.98 |
| | W/NaZSM-5 (1:0.3) | | | | |
| | 4.3 wt % of cellulose | | | | |
| | 220° C., 40 bar 6 hrs, 1000 rpm | | | | |
| 23. | 5% Al-8% Ni-25% | 100 | 92.1 | 0.7 | 92.8 |
| | W/NaZSM-5 220° C., | | | | |
| | 40 bar, 12 hrs, 1000 rpm | | | | |

Table 2 below summarizes the results obtained with preferred catalyst systems providing high yields of glycols:

TABLE 2

| | | (%) | Selectivity | | |
|---|---|---|---|---|---|
| Sr. | | conver- | | | Total |
| No. | Catalysts | sion | EG | 1,2-PD | glycol % |
| 1. | 7% Al-8% Ni- | 100 | 68.06 | 4.13 | 72.19 |
| | 20% W/NaZSM-5 (1:1); | | | | |
| | 220° C., 40 bar 6 hrs, | | | | |
| | 1000 rpm | | | | |
| 2. | 5% Al-8% Ni- | 100 | 69.55 | 5.44 | 74.99 |
| | 20% W/NaZSM-5; 220° C., | | | | |
| | 40 bar 6 hrs, 1000 rpm | | | | |
| 3. | 8% Ni-15% W/NaZSM-5; | 100 | 50.56 | 6.61 | 57.17 |
| | 220° C., 40 bar 6 hrs, | | | | |
| | 1000 rpm | | | | |
| 4. | 5% Al-8% Ni- | 100 | 73.92 | 5.66 | 79.58 |
| | 20% W/NaZSM-5 (′1:1); | | | | |
| | 220° C., 40 bar 6 hrs, | | | | |
| | 1000 rpm | | | | |
| 5. | 7% Al-8% Ni- | 100 | 68.06 | 4.13 | 72.19 |
| | 20% W/NaZSM-5 (1:1); | | | | |
| | 220° C., 40 bar 6 hrs, | | | | |
| | 1000 rpm | | | | |
| 6. | 7% Al-8% Ni- | 100 | 63.03 | 4.61′ | 67.64 |
| | 20% W/NaZSM-5; 220° C., | | | | |
| | 40 bar 6 hrs, 1000 rpm | | | | |
| 7. | 5% Al-6% Ni- | 100 | 63.88 | 3.75 | 67.63 |
| | 10% W/NaZSM-5; 220° C., | | | | |
| | 40 bar 6 hrs, 1000 rpm | | | | |
| 8. | 8% Ni-20% W/NaZSM-5; | 100 | 53.44 | 3.50 | 56.94 |
| | 220° C., 40 bar 6 hrs, | | | | |
| | 1000 rpm | | | | |
| 9. | 5% Al-8% Ni- | 100 | 73.31 | 8.94 | 82.25 |
| | 25% W/NaZSM-5; 220° C., | | | | |
| | 40 bar 6 hrs, 1000 rpm | | | | |
| 10. | 5% Al-8% Ni- | 100 | 76.71 | 7.54 | 84.25 |
| | 25% W/NaZSM-5 (1:1); | | | | |
| | 220° C., 40 bar 6 hrs, | | | | |
| | 1000 rpm | | | | |
| 11. | 5% Al-8% Ni- | 100 | 61.85 | 8.34 | 70.19 |
| | 25% W/NaZSM-5; 220° C., | | | | |
| | 40 bar, 1.5 hrs, 1000 rpm | | | | |
| 12. | 5% Al-8% Ni- | 100 | 62.56 | 19.42 | 81.98 |
| | 25% W/NaZSM-5 | | | | |
| | (1:0.3)4.3 wt % of cellulose | | | | |
| | 220° C., 40 bar 6 hrs, | | | | |
| | 1000 rpm | | | | |
| 13. | 5% Al-8% Ni- | 100 | 92.1 | 0.7 | 92.8 |
| | 25% W/NaZSM-5 | | | | |
| | 220° C., 70 bar, 12 hrs, | | | | |
| | 1000 rpm | | | | |

A surprisingly feature of the process of the present invention is that at about 4 wt % of cellulose, lower catalyst concentration (1:0.3) provides total glycol of 82%, providing a significant industrial advantage which is a non-obvious feature to the present invention.

The catalyst is reusable giving good selectivity towards ethylene glycol. Study on reusability of the catalyst is done by using 5% Al-8% Ni-25% W/ZSM-5 catalyst being a most preferred catalyst. As the catalyst system of 5% Al-8% Ni-25% W/ZSM-5 gives maximum EG selectivity of 92.1% with 100% conversion of cellulose. Table 3 below shows the reusability (studied at 40 bar H₂, 220° C., 6 hrs, 1000 rpm) results of the preferred catalyst 5% Al-8% Ni-25% W/ZSM-5, which shows that the catalyst can be reused for three more times.

TABLE 3

| | | % selectivity | | |
|---|---|---|---|---|
| Run | | | | Total |
| No. | Conversion | EG | 1,2-PG | Glycols |
| Run 0 | 100% | 76.71 | 7.54 | 84.25 |
| Run 1 | 100% | 75.93 | 9.54 | 85.47 |
| Run 2 | 100% | 72.92 | 9.17 | 82.09 |
| Run 3 | 100% | 66.91 | 12.91 | 79.82 |

The scale up reaction with higher concentration of cellulose on less amount of catalyst is also tried and it is found that cellulose conversion is remained 100% with ˜82% yield of glycol which is highly significant for commercialization process.

### General Information

HPLC analysis is carried out in Perkin Elmer series 500 instrument using manual injection and the data collection is done using RID and TC NAV software. The column using for the analysis is Rezex Organic Acid Column with 0.005M H₂SO₄ in millipore water as mobile phase. The run time of analysis is 40-60 minutes at 0.5 ml min⁻¹ flow rate and column temperature of 60° C.

### EXAMPLES

Following examples are given by way of illustration and therefore should not be construed to limit the scope of the invention.

### Example 1: General Process for the Preparation of the Catalyst

Metal precursor-1 (Aluminium nitrate nonahydrate)+metal precursor-2 (Ammonium meta tungstate) was dissolved in DI water in a beaker I and metal precursor-3 (Nickel nitrate hexahydrate) is separately dissolved in DI water in beaker II by stirring. Both the solutions are simultaneously added on to the support at 80° C. under stirring for 6-8 hours. The mixture is sonicated couple of times in between for 30 minutes each. A little amount of DI water is added in between to make up with the loss of water, if any. After 8 hours, the mixture is kept for drying in oven at 110° C. for 10 to 12 hr. The dried catalyst was ground into fine powder and calcined in Muffle furnace at 550° C. with the ramp rate of 2° C. min⁻¹ for 4 hours. The calcined sample was then reduced under hydrogen atmosphere in tubular furnace at 400° C. with ramp rate of 5° C. min⁻¹ for 4 hours. The reduced sample is used for the reaction.

### Example 2: Conversion of Cellulose into Value Added Products

The activity of the prepared catalyst on cellulose conversion is studied on 50 ml Parr SS Batch Reactor (5500 series with 4848 Controller). The catalyst and cellulose were taken at the ratio of 1:1 in Parr reactor with sufficient amount of water to make it 1 wt % of cellulose solution. The system was first purged with Nitrogen and the with Hydrogen gas. After purging, the reactor was pressurized to 40 bar pressure at 25° C. and heated upto 220° C. (200-230° C. based on reaction requirements). Then, 70 bar reaction pressure was applied and maintained with constant stirring (700-1000 rpm based on reaction requirements) for a period of 1.5-12 hr. The active result is shown by the reaction condition for a period of 12 hours and 6 hours by the catalyst 5% Al-8% Ni-25% W/Na7SM-5 at 220° C., 70 bar pressure at reaction temperature.

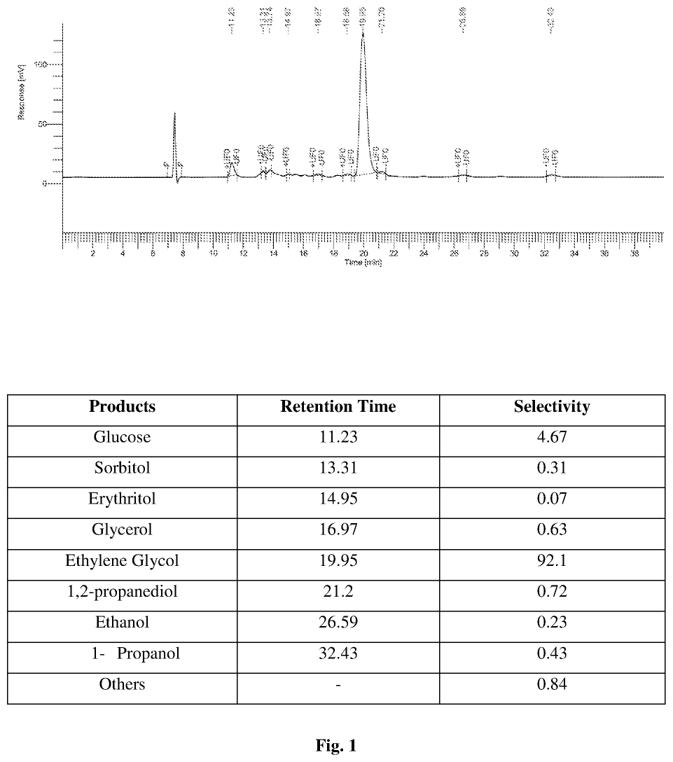

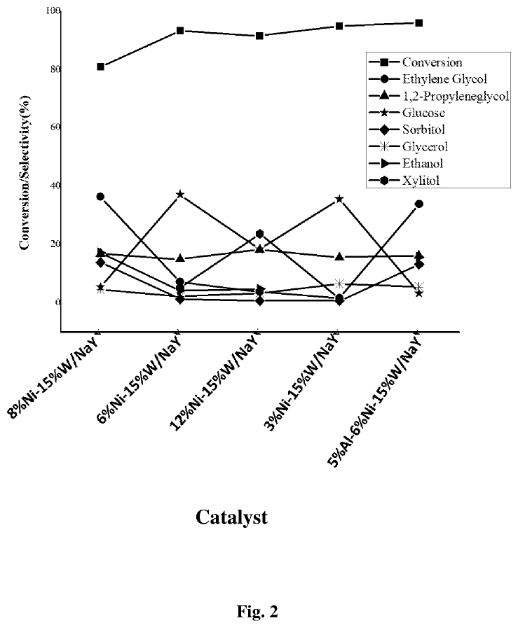

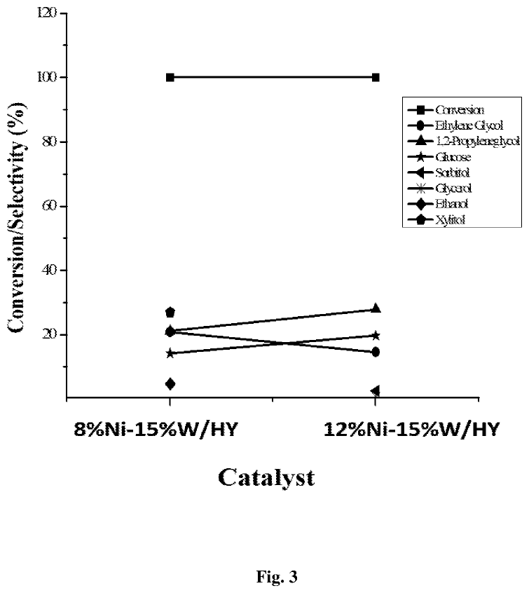

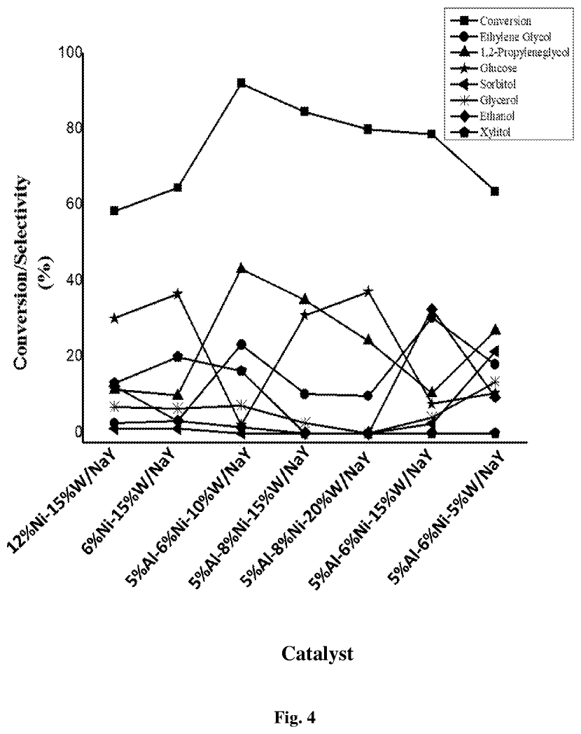

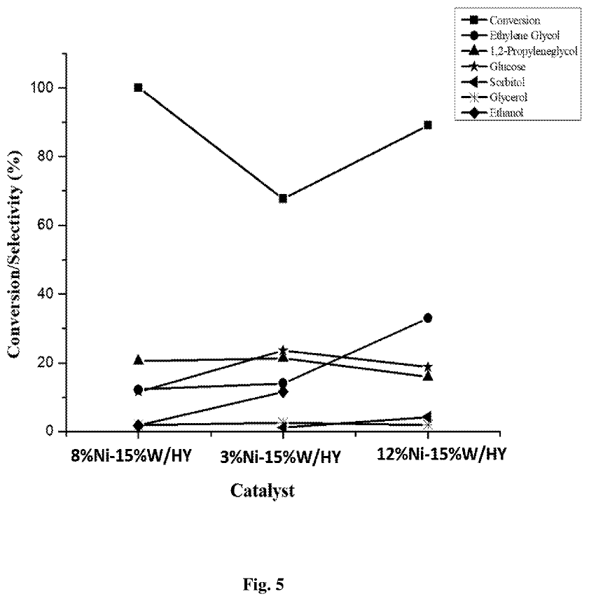

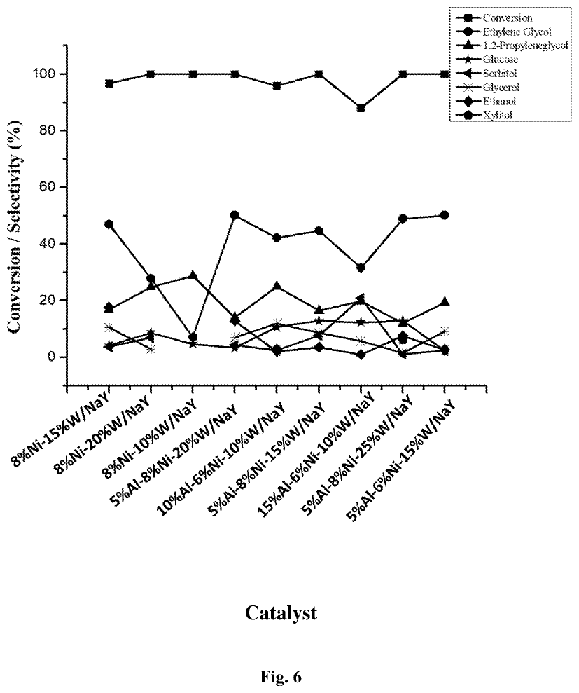

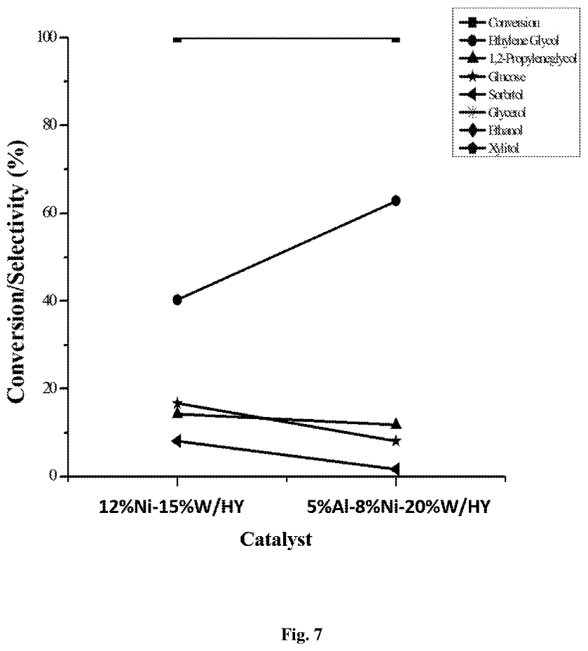

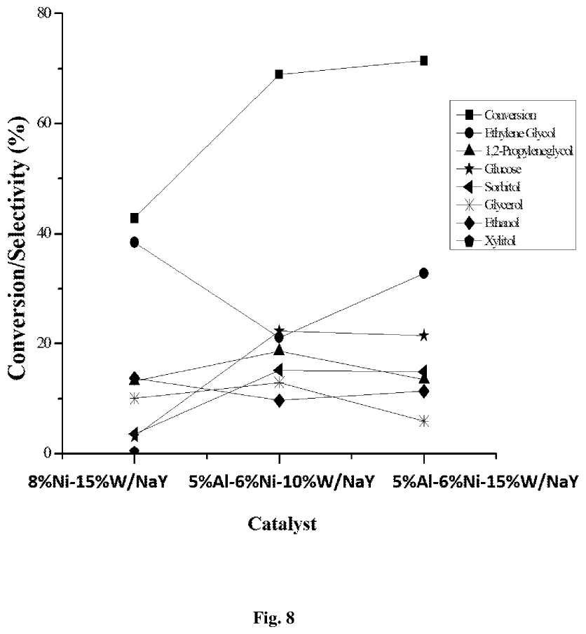

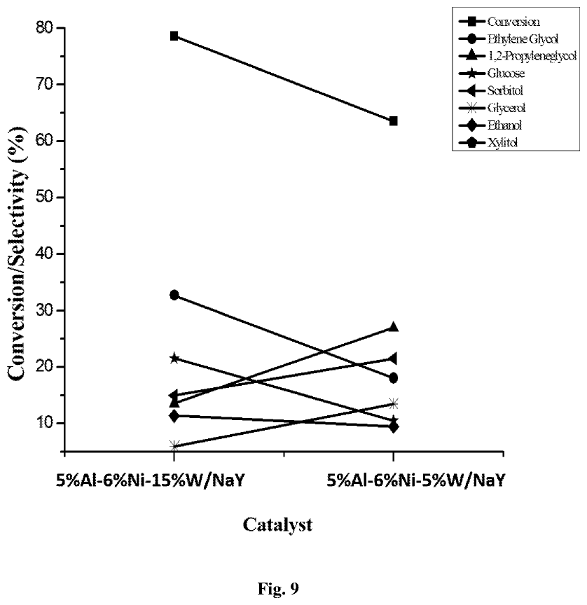

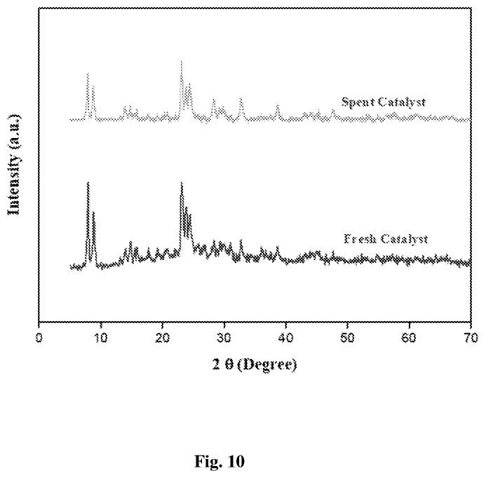

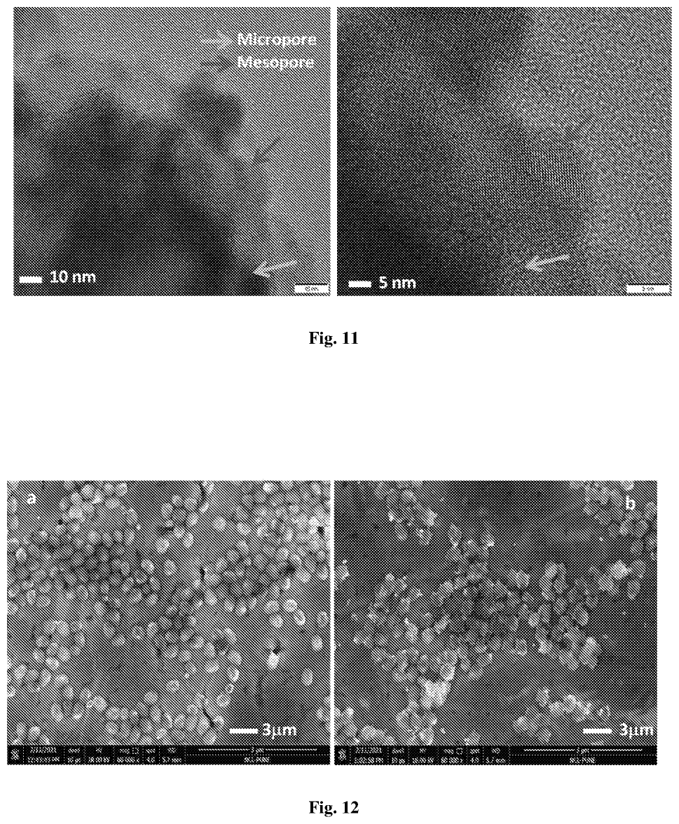

After the reaction completed, the stirring and heating was switched off and the system was allowed to cool by its own. The product mixture was then filtered, the catalyst was recovered and the product solution was analyzed using HPLC (FIG. 1).

### Example 3: Conversion of Bio-Cellulose [Sugarcane Bagasse] into Value Added Products

The catalytic activity was tested as listed in example 2 with sugarcane bagasse as substrate in place of cellulose. 100% conversion of bagasse was observed with selectivity around 70% towards 1,2-PD+EG and 13% towards glucose.

### Advantages of the Invention

Less expensive metal precursors are employed
Catalyst preparation is by simple process of impregnation
Catalyst preparation is by environmentally friendly process
100% conversion of cellulose and high yield of valuable products at low temperature and optimum pressure is obtained.
Catalyst is reusable
Support is not damaged even after several runs
Negligent metal leaching in reruns
At higher concentration of cellulose, lower catalyst composition obtained higher EG selectivity.

1. A process for conversion of cellulose into polyols comprising the steps of:
reacting cellulose with a catalyst in the ratio ranging between 1:0.3 to 1:1 in a batch reactor with a solvent, at a temperature in the range of 200° C. to 230° C., pH in the range of 4.5 to 2 with stirring in the range of 700 to 1000 rpm, and pressure in the range of 40 to 70 bars of Hydrogen for a period in the range of of 1.5-12 hrs to obtain polyol with 100% conversion of cellulose to polyols; wherein the polyol is ethylene glycol, 1,2-propane diol, glucose, sorbitol, xylitol erythritol and glycerol; and
the catalyst is selected from the group consisting of Al—Ni—W/HY, Al—Ni—W/NaY Al—Ni—W/HZSM-5 and Al—Ni—W/Na-ZSM-5 having the ratio of metal Ni in the range of 3-12%, Al in the range of 5-15% and Win the range of 5-30%.

2. A catalyst for 100% conversion of cellulose in polyol comprising:
Al—Ni—W, wherein the Al is in the range of 5%-15%; Ni is in the range of 3%-12%; and W is in the range of 5%-30%; and
a support adding up to 100%, wherein the support is selected from H-ZSM-S, Na-ZSM-S, Y or beta zeolite; HY zeolite, NaY Zeolite, gamma Alumina or boehmite.

3. The catalyst as claimed in claim 2, wherein the catalyst 5% Al-8% Ni-25% W/Na-ZSM-5 provides ethylene glycol (EG) selectivity of 89-92.1%.

4. The catalyst as claimed in claim 3, wherein the catalyst 5% Al-8% Ni-25% W/Na-ZSM-5 is recyclable.

5. A process for preparation of the catalyst as claimed in claim 2, wherein said catalyst is prepared by wet impregnation method comprising the steps of:
a) simulataneously adding solutions of metal precursors in a solvent on a support under stirring at a temperature in the range of 60-85° C. for a period of time in the range of 6-8 hrs to obtain a first mixture;
b) drying the first mixture as obtained in step (a) in an oven at a temperature in the range of 80-120° C. for a period of time in the range of 10-12 hrs to obtain a second mixture;
c) grinding and calcining the second mixture as obtained at step (b) at a temperature in the range of 500-600° C. for a period in the range of 4 to 5 hrs followed by reducing under hydrogen at a temperature in the range of 350-450° C. for a period in the range of 4 to 5 hrs to obtain the catalyst.

6. The process as claimed in claim 5, wherein the support in step (a) is selected from ZSM-5, Na-ZSM-5, Y or beta zeolite; HY zeolite, NaY Zeolite, gamma Alumina or boehmite, preferably ZSM-5 or Y.

7. The process as claimed in claim 5, wherein said metal precursors in step (a) are Aluminium nitrate nonahydrate, Ammonium metatungstate and Nickel nitrate hexahydrate.

8. The process as claimed in claim 5, wherein the cellulose is selected from pure cellulose or bio-cellulose.

9. The process as claimed in claim 5, wherein the solvent is water.

10. The process as claimed in claim 1, wherein the solvent is water.
